(19) United States
(12) Patent Application Publication
(10) Pub. No.: US 2010/0285550 A1
(43) Pub. Date: Nov. 11, 2010

(54) Delignification of Lignocellulose-Containing Material

(75) Inventor: Yongming Zhu, Wake Forst, NC (US)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/811,986

(22) PCT Filed: Jan. 9, 2009
(86) PCT No.: PCT/US09/30585

§ 371 Date: Jul. 7, 2010

Related U.S. Application Data
(60) Provisional application No. 61/020,473, filed on Jan. 11, 2008.

(51) Int. Cl. C12P 7/16; C12P 1/00; D21C 3/00; C12P 7/10

(52) U.S. Cl. 435/160; 435/41; 162/1; 162/90; 162/77; 162/72; 435/165

(57) ABSTRACT

The invention relates to processes of delignifying lignocellulose-containing material, wherein the lignocellulose-containing material is treated with a delignification catalyst and a lignin solubilizing agent.

### TECHNICAL FIELD

The present invention relates to methods of delignifying lignocellulose-containing material. The invention also relates to processes of producing a fermentation product from such delignified material using a fermenting organism. Treating solutions which may suitably be used in delignification methods of the invention and the use of such treating solutions are also described.

### BACKGROUND ART

Due to the limited reserves of fossil fuels and worries about emission of greenhouse gasses there is an increasing focus on using renewable energy sources. Commercial production of biofuel (mainly ethanol) and other fermentation products from starch and sugars is already ongoing, but the production cost is relatively high primarily because grains and sugar crops are expensive feedstocks. Therefore, the attention has turned towards the cheaper lignocellulose feedstocks (i.e., biomass) such as agricultural residues, grasses, and wood.

Processes for producing biofuels from lignocellulose-containing material are described in the art and conventionally include the steps of pretreatment, hydrolysis, and fermentation. As the structure of lignocellulose is not directly accessible to enzymatic hydrolysis, partly due to the crystaine structure of cellulose and the presence of a lignin seal pre-treatment is necessary.

Methods of delignifying lignocellulose-containing material are known in the art. For instance, delignification using ammonia reduces the lignin content and causes solubilisation of the hemicellulose and lignin fractions and enhances enzyme accessibility to cellulose. After delignification, cellulose and hemicellulose can be hydrolyzed enzymatically, e.g., by cellulolytic and hemicellulolytic enzymes, to convert the carbohydrate polymers into fermentable sugars which may be fermented into a desired fermentation product, such as ethanol.

Even though methods of removing lignin from lignocellulose-containing materials are known in the art there is still a need for improved delignification methods.

### SUMMARY OF THE INVENTION

The object of the present invention is to provide more efficient and/or cost efficient delignification methods suitable for use in fermentation product production processes, especially biofuel production processes.

In the first aspect the present invention relates to methods of delignifying lignocellulose-containing material, wherein lignocellulose-containing material is treated with a delignification catalyst and a lignin solubilizing agent.

In the second aspect the invention provides processes of producing a fermentation product from lignocellulose-containing material comprising the steps of:

(a) delignifying lignocellulose-containing material using a delignification method of the invention;

(b) hydrolyzing the material;

(c) fermenting using a fermenting organism.

In the third aspect the invention relates to treating soiutions for delignifying lignocellulose-containing material comprising;

i) from 1-30 wt. % delignification catalyst; and

ii) from 5-60 wt. % lignin solubilizing agent.

In the fourth aspect the invention relates to use of a treating solution of the invention for delignification of lignocellulose-containing material.

### BRIEF DESCRIPTION OF THE FIGURES

FIG. 1 summarizes the results of ammonia/ethanol treatment of corn stover at 140° C.

FIG. 2 shows the effect of ethanol on carbohydrates and lignin retention in wheat straw at 120° C.

FIG. 3 shows the effect of ammonia concentration and processing time on carbohydrates and lignin retention in wheat straw.

FIG. 4 shows the effect of ammonia/ethanol treatment on enzymatic hydrolysis of corn stover,

### DETAILED DESCRIPTION OF THE INVENTION

The present invention relates to methods of delignifying lignocellulose-containing material. According to the invention lignin is effectively removed, while a substantial part of the cellulose and hemicellulose is retained. The retained carbohydrate polymers and fermentable sugars may be used for producing fermentation products such as biofuel products including especially ethanol and butanol. Examples of other fermentation products can be found below in the section “Fermentation Products.” The invention also provides processes for producing desired fermentation products from delignified lignocellulose-containing material by hydrolysis and fermentation using a suitable fermenting organism.

The inventors found that when utilizing a mixture of ammonia and ethanol for treating lignocellulose-containing material under relatively mild temperatures (preferably around 140° C.) the carbohydrate yield was improved and the removal of lignin was promoted compared to, e.g., corresponding methods only using ammonia. Ammonium serves as catalyst for delignification and ethanol facilitates the solubilisation and removal of lignin from the solid phase, in addition, not being bound by any particular theory, the ethanol addition to the reaction system is believed to help the preservation of carbohydrates in the solids probably by reducing their solubility.

In the first aspect the invention relates to methods of delignifying lignocellulose-containing material, wherein lignocellulose-containing material is treated with a delignification catalyst and a lignin solubilizing agent.

In an embodiment of the invention the lignocellulose-containing material is treated in a treating solution comprising a delignification catalyst and a lignin solubilizing agent.

In a preferred embodiment the treating solution is an aqueous treating solution. in another embodiment, delignification is carried out in an aqueous slurry comprising lignocellulose-containing material and water, and further comprising a delignification catalyst and a lignin solubilizing agent.

In another embodiment, the temperature during treatment is in the range from 60-180° C., preferably 120-160°, especially around 140° C. The delignification treatment is typically carried out for 10 minutes to 1 week, preferably 30 minutes to 6 hours, such as 1 hour to 12 hours, Delignification treatment is typically carried out at alkaline pH, such as at a pH in the range from 8-12. The lignocellulose-containing material typically constitutes from 5-30 wt. %, preferably in the range 10-25 wt. % of the treating solution. Examples of lignocellulose-containing material can be found below in the section “Lignocellulose-containing material (Biomass).” In a preferred embodiment, the material is non-wood lignocellulose-containing material, such as corn stover and/or wheat straw.

### Delignification Catalysts

According to the invention the delignification catalyst may be any suitable delignification catalyst. In a preferred embodiment, the catalyst is ammonium such as aqueous ammonium.

In a preferred embodiment, the lignocellulose-containing material is subjected to 0.02-40 g ammonium per g lignocellulose substrate. In general the delignification catalyst may be present during treatment in a concentration in the range from 1-30 wt. %, preferably 2-10 wt. %, especially around 5 wt. % per g treating solution. In a preferred embodiment ammonium is dosed so that the treating solution comprises from 0.02-40 g ammonia per g lignocellulose substrate.

Lignin Solubilizing Agents

According to the invention the lignin solubilizing agent may be any suitable lignin solubilizing agent, such as organic alcohols, preferably ethanol: glycerol; or acetone, in general the delignification catalyst may according to the invention be present during delignification treatment in a concentration in the range from 5-60 wt. %, preferably 30-50 wt, %, especially around 40 wt. % per g treating solution. In a preferred embodiment ethanol is dosed so that the treating solution comprises from 0.05-10 g ethanol per g lignocelluloses substrate.

Production of Fermentation Products from Lignocellulose-Containing Material (Biomass)

In one aspect of the invention, processes of producing desired fermentation products from lignocellulose-containing material are provided. Lignocellulose-containing materials primarily consist of cellulose, hemicellulose, and lignin and are often referred to as “biomass.”

The invention relates to processes of producing a fermentation product from lignocellulose-containing material, comprising the steps of.

(a) delignifying lignocellulose-containing material using a delignification method of the invention;

(b) hydrolyzing the material; and

(c) fermenting using a fermenting organism.

According to the invention, delignification is carried out in accordance with the delignification method of the invention as described above. However, it is to be understood that in addition, the lignocelluloses material may be treated in a suitable way before delignification in step (a), e.g., by subjecting the lignocellulose-containing material to another suitable chemical and/or mechanical pre-treatment step. Such pre-treatment steps are well-known in the art. in another embodiment, the material is reduced in particle size, e.g., by milling. In another embodiment, steps (b) and (c) are carried out simultaneously or sequentially. Examples of delignification catalysts and solubilisation agents are described above. In a preferred embodiment, the lignocellulose-containing material is treated with ammonium, such as aqueous ammonium, as a delignification catalyst and ethanol as lignin solubilizing agent in step (a). In a preferred embodiment the lignocellulose-containing material is delignified in step (a) by treating the material with from 0.02-40 g aqueous ammonium per g lignocellulose substrate in combination with from 0.05-10 g ethanol per g lignocelluloses substrate. Initially (i.e., before delignification) a slurry comprising lignocellulose-containing material and treating solution is prepared. The slurry may be prepared, e.g., by adding the lignocellulose-containing material to the treating solution or by adding the treating solution to the lignocelluloses-containing material. Delignification is carried out in said slurry wherein the lignocellulose-containing material constitutes from 5-30 wt. %, preferably from 10-25 wt. %. The temperature during delignification in step (a) may be in the range from 60-180° C., preferably 120-160° C., especially around 140° C. in another embodiment, delignification in step (a) is carried out for 10 minutes to 1 week, preferably 1 hour to 12 hours. In an embodiment the delignification catalyst is dosed so that it comprises from 1-30 wt. %, preferably 2-10 wt. %, especially around 5 wt. % per g treating solution. In a preferred embodiment the lignin solubilizing agent is dosed so that the concentration is in the range from 5-60 wt. %, preferably 30-50 wt. %, especially around 40 wt. % per g treating solution.

In a preferred embodiment, hydrolysis in step (b) and fermentation in step (c) are carried out as simultaneously hydrolysis and fermentation process (SSF process) or a hybrid hydrolysis and fermentation process (HHF process). Hydrolysis, SSF or HHF is carried out using a cellulolytic enzyme or hemicellulolytic enzyme, or a combination thereof. Examples of hydrolytic enzymes can be found in the “Enzymes” section below.

The fermenting organism used in step (c), SSF, or HHF is typically of microbial origin, preferably yeast origin, preferably a strain of the genus Saccharomyces, Pichia, or Kluyveromyces. However, fermentation organisms of, e.g., bacterial origin is also contemplated. A non-exhaustive list of fermenting organisms can be found below in the section “Fermentation Organisms,” In a preferred embodiment the fermentation product is a biofuel, such as especially an alcohol, such as ethanol or butanol.

### Hydrolysis

According to the invention the delignified lignocellulose-containing material is hydrolyzed. In a preferred embodiment hydrolysis is carried out enzymatically using a hydrolytic enzyme or mixture of hydrolytic enzymes. According to the invention the delignified lignocellulose-containing material, to be fermented, is hydrolyzed by one or more hydrolases (class EC 3 according to the Enzyme Nomenclature), preferably one or more carbohydrases selected from the group consisting of cellulase, hemicellulase, or amylase, such as alpha-amylase, maltogenic amylase or beta-amylase. A protease may also be present.

The enzymes used for hydrolysis are capable of directly or indirectly converting carbohydrate polymers (e.g., cellulose and/or hemicellulose) into fermentable sugars which can be fermented into a desired fermentation product, such as ethanol.

In a preferred embodiment the carbohydrase has cellulolytic enzyme activity. Suitable carbohydrases are described in the “Enzymes” section below.

Hemicellulose polymers can be broken down by hemicellulases and/or acid hydrolysis to release its five and six carbon sugar components. The six carbon sugars (hexoses), such as glucose, galactose and mannose, can readily be fermented to. e.g., ethanol, acetone, butanol, glycerol, citric acid, fumaric acid etc. by suitable fermenting organisms including yeast. Preferred for ethanol fermentation is yeast of the species Saccharomyces cerevisiae, preferably strains which are resistant towards high levels of ethanol, i.e., up to, e.g., about 10, 12, 15 or 20 vol. % or more ethanol.

In a preferred embodiment the delignified lignocellulose-containing material is hydrolyzed using a hemicellulase, preferably a xylanase, esterase, cellobiase, or combination of two or more thereof.

Hydrolysis may also be carried out in the presence of a combination of hemicellulases and/or cellulases, and optionally one or more of the other enzyme activities mentioned above.

The enzymatic treatment may be earned out in a suitable aqueous environment under conditions which can readily be determined by one skilled in the art. In a preferred embodiment hydrolysis is carried out at optimal conditions for the enzyme(s) in question.

Suitable process time, temperature and pH conditions can readily be determined by one skilled in the art. Preferably, hydrolysis is carried out at a temperature between 30 and 70° C., preferably between 40 and 60° C., especially around 50° C. The process is preferably carried out at a pH in the range from 3-8, preferably pH 4-6, especially around pH 5, Preferably, hydrolysis is carried out for between 8 and 72 hours, preferably between 12 and 48 hours, especially around 24 hours.

### Fermentation of Lignocellulose Derived Material

Fermentation of delignified lignocellulose-containing material may be carried out in any suitable way. According to the invention hydrolysis in step (b) and fermentation in step (c) may be carried out simultaneously (SSF). sequentially (SHF), or as hybrid hydrolysis and fermentation (HHF).

### SSF, HHF and SHF

In one embodiment of the present invention, hydrolysis and fermentation is carried out as a simultaneous hydrolysis and fermentation step (SSF). In general this means that combined/simultaneous hydrolysis and fermentation are carried out at conditions (e.g., temperature and/or pH) suitable, preferably optimal, for the fermenting organism(s) in question.

In another embodiment hydrolysis step and fermentation step are carried out as hybrid hydrolysis and fermentation (HHF). HHF typically begins with a separate partial hydrolysis step and ends with a simultaneous hydrolysis and fermentation step. The separate partial hydrolysis step is an enzymatic cellulose saccharification step typically carried out at conditions (e.g., at higher temperatures) suitable, preferably optimal, for the hydrolyzing enzyme(s) in question. The subsequent simultaneous hydrolysis and fermentation step is typically carried out at conditions suitable for the fermenting organism(s) (often at lower temperatures than the separate hydrolysis step).

In another embodiment, the hydrolysis and fermentation steps may also be carried out as separate hydrolysis and fermentation, where the hydrolysis is taken to completion before initiation of fermentation. This is often referred to as “SHF”.

### Fermenting Organisms

The term “fermenting organism” refers to any organism, including bacterial and fungal organisms, including yeast and filamentous fungi, suitable for producing a desired fermentation product. The fermenting organism may be C6 or C5 fermenting organisms, or a combination thereof. Both C6 and C5 fermenting organisms are well known in the art.

Suitable fermenting organisms according to the invention are able to ferment, i.e., convert fermentable sugars, such as glucose, fructose maltose, xylose, mannose or arabinose, directly or indirectly into the desired fermentation product.

Examples of fermenting organisms include fungal organisms such as yeast. Preferred yeast includes strains of the genus Saccharomyces, in particular strains of Saccharomyces cerevisiae or Saccharomyces uvarum: a strain of Pichia, preferably Pichia stipitis such as Pichia stipitis CBS 5773 or Pichia pastoriss; a strain of the genus Candida, in particular a strain of Candida utilis, Candida arabinofermentans, Candida diddensii, Candida sonorensis, Candida shehatae, Candida tropicalis, or Candida boidinii. Other fermenting organisms include strains of Hansenula, in particular Hansenula polymorpha or Hansenula anomala: Kluyvermyces, in particular Kluyveromyces fragilis or Kluyvermyces marxianus; and Schizosaccharomyces, in particular Schizosaccharomyces pombe.

Preferred bacterial fermenting organisms include strains of Escherichia, in particular Escherichia coli, strains of Zymomonas, in particular Zymomonas mobilis, strains of Zymobacter, in particular Zyrmbactor palmae, strains of Klebsiella in particular Klebsiella oxytoca, strains of Leuconostoc, in particular Leuconostoc mesenteroides, strains of Clostridium, in particular Clostridium butyricum, strains of Enterobacter, in particular Enterobacter aerogenes and strains of Thermoanaerobacter, in particular Thermoanaerobacter BG1L1 (Appl. Microbiol Biotech. 77: 61-86) and Thermoanarobacter ethanolicus, Thermoanaerobacter thermosaccharolyticum, or Thermoanaerobacter mathranii. Strains of Lactobacillus are also envisioned as are strains of Corynebactehum glutamicum R. Bacillus thermogiucosidaisus, and Geobacillus thermoglucosidasius.

In an embodiment the fermenting organism is a C6 sugar fermenting organism, such as a strain of, e.g., Saccharomyces cerevisiae.

In connection with especially fermentation of lignocellulose derived materials, C5 sugar fermenting organisms are contemplated. Most C5 sugar fermenting organisms also ferment C6 sugars. Examples of C5 sugar fermenting organisms include strains of Pichia, such as of the species Pichia stipitis. C5 sugar fermenting bacteria are also known. Also some Saccharomyces cerevisae strains ferment C5 (and C6) sugars. Examples are genetically modified strains of Saccharomyces spp that are capable of fermenting C5 sugars include the ones concerned in, e.g., Ho et al., 1998, Applied and Environmental Microbiology, p. 1852-1859 and Karhumaa et al., 2006, Microbial Cell Factories 5:18, and Kuyper et al., 2005, FEMS Yeast Research 5: 925-934.

In one embodiment the fermenting organism is added to the fermentation medium so that the viable fermenting organism, such as yeast, count per mL of fermentation medium is in the range from 10⁵ to 10¹², preferably from 10⁷ to 10¹⁰, especially about 5×10⁷.

Commercially available yeast includes, e.g., RED STAR™ and ETHANOL RED™ yeast (available from Fermentis/Lesaffre, USA). FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties).

According to the invention the fermenting organism capable of producing a desired fermentation product from fermentable sugars, including glucose, fructose maltose, xylose, mannose, and/or arabinose, is preferably grown under precise conditions at a particular growth rate. When the fermenting organism is introduced into/added to the fermentation medium the inoculated fermenting organism pass through a number of stages. Initially growth does not occur. This period is referred to as the “lag phase” and may be considered a period of adaptation. During the next phase referred to as the “exponential phase” the growth rate gradually increases. After a period of maximum growth the rate ceases and the fermenting organism enters “stationary phase”. After a further period of time the fermenting organism enters the “death phase” where the number of viable cells declines.

### Fermentation Products

The term “fermentation product” means a product produced by a process including a fermentation step using a fermenting organism. Fermentation products contemplated according to the invention include alcohols (e.g., ethanol, methanol, butanol); organic acids (e.g., citric acid, acetic acid, itaconic acid, Sactic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. Preferred fermentation processes used include alcohol fermentation processes. The fermentation product, such as ethanol, obtained according to the invention, may preferably be used as biofuel. However, in the case of ethanol if may also be used as potable ethanol.

### Fermentation of Lignocellulose-derived Sugars

As mentioned above different kinds of fermenting organisms may be used for fermenting sugars derived from delignified lignocellulose-containing materials. Fermentations are typically carried out by yeast, bacteria or filamentous fungi, including the ones mentioned in the “Fermenting Organisms” section above. If the aim is C6 fermentable sugars the conditions are usually similar to starch fermentations as described above. However, if the aim is to ferment C5 sugars (e.g., xylose) or a combination of C6 and C5 fermentable sugars the fermenting organism(s) and/or fermentation conditions may differ.

Bacteria fermentations may be carried out at higher temperatures, such as up to 75° C., e.g., between 40-7° C., such as between 50-60° C., than conventional yeast fermentations, which are typically carried out at temperatures from 20-40° C. However, bacteria fermentations at temperature as low as 20° C. are also known. Fermentations are typically carried out at a pH in the range between 3 and 7, preferably from pH 3,5 to 6, such as around pH 5. Fermentations are typically ongoing for 24-96 hours.

### Recovery

Subsequent to fermentation the fermentation product may be separated from the fermented slurry. The slurry may be distilled to extract the desired fermentation product or the desired fermentation product may be extracted from the fermented slurry by micro or membrane filtration techniques. Alternatively the fermentation product may be recovered by stripping. Methods for recovery are well Known in the art.

### Lignocellulose-containing Material (Biomass)

Any suitable lignocellulose-containing material is contemplated in context of the present invention. Lignocellulose-containing material may be any material containing lignocellulose. in a preferred embodiment the lignocellulose-containing material contains at least 50 wt. %, preferably at least 70 wt. %, more preferably at least 90 wt. % lignocellulose. If is to be understood that the lignocellulose-containing material may also comprise other constituents such as cellulosic material, such as cellulose, hemicellulose, and may also comprise constituents such as sugars, such as fermentable sugars and/or un-fermentable sugars.

Ligno-celluiose-containing material is generally found, for example, in the stems, leaves, hulls, husks, and cobs of plants or leaves, branches, and wood of trees, Lignocellulosic material can also be, but is not limited to, herbaceous material, agricultural residues, forestry residues, municipal solid wastes, waste paper, and pulp and paper mill residues. It is understood herein that lignocellulose-containing material may be in the form of plant cell wall material containing lignin, cellulose, and hemi-cellulose in a mixed matrix.

In an embodiment the lignocellulose-containing material is corn fiber, rice straw, pine wood, wood chips, poplar, wheat straw, switchgrass, bagasse, paper and pulp processing waste.

Other more specific examples include corn stover, corn cobs, corn fiber, hardwood such as poplar and birch, softwood, cereal straw such as wheat straw, switch grass, Miscanthus, rice hulls, municipal solid waste (MSW), industrial organic waste, office paper, or mixtures thereof.

In a preferred embodiment the lignocellulose-containing material is corn stover or corn cobs. In another preferred embodiment, the lignocellulose-containing material is corn fiber. In another preferred embodiment, the lignocellulose-containing material is switch grass. In another preferred embodiment, the lignocellulose-containing material is bagasse.

### Enzymes

Even if not specifically mentioned in context of a process of the invention, it is to be understood that the enzymes are used in an effective amount.

### Cellulolytic Enzymes

One or more celluloytic enzymes may be present during fermentation, SSF, or HHF. The terms “cellulolytic enzymes” as used herein are understood as comprising the cellobiohydrolases (EC 3.2.1.91), e.g., cellobiohydrolase I and cellobiohydrolase II, as well as the endo-glucanases (EC 3.2.1.4) and beta-glucosidases (EC 3.2.1.21).

In order to be efficient, the digestion of cellulose may require several types of enzymes acting cooperatively. At least three categories of enzymes are often needed to convert cellulose into glucose: endoglucanases (EC 3.2.1.4) that cut the cellulose chains at random: cellobiohydrolases (EC 3.2.1.91) which cleave cellobiosyl units from the cellulose chain ends and beta-glucosidases (EC 3.2.1.21) that convert cellobiose and soluble cellodextrins into glucose. Among these three categories of enzymes involved in the biodegradafion of cellulose, cellobiohydrolases are the key enzymes for the degradation of native crystalline cellulose. The term “cellobiohydrolase I” is defined herein as a cellulose 1,4-beta-cellobiosidase (also referred to as Exo-glucanase, Exo-cellobiohydrolase or 1,4-beta-cellobiohydrolase) activity, as defined in the enzyme class EC 3.2.1.91, which catalyzes the hydrolysis of 1,4-beta-D-glucosidic linkages in cellulose and cellotetraose, by the release of cellobiose from the non-reducing ends of the chains. The definition of the term “cellobiohydrolase II activity” is identical, except that cellobiohydrolase II attacks from the reducing ends of the chains.

The cellulolytic enzyme may comprise a carbohydrate-binding module (CBM) which enhances the binding of the enzyme to a lignocellulose-containing fiber and increases the efficacy of the catalytic active part of the enzyme. A CBM is defined as contiguous amino acid sequence within a carbohydrate-active enzyme with a discreet fold having carbohydrate-binding activity. For further information of CBMs see the CAZy internet server (Supra) or Tomme et al. (1995) in Enzymatic Degradation of Insoluble Polysaccharides (Saddler and Penner, eds.), Cellulose-binding domains: classification and properties, pp. 142-163, American Chemical Society, Washington.

In a preferred embodiment the cellulases or cellulolytic enzymes may be a cellulolytic preparation as defined PCT/2008/065417, which is hereby incorporated by reference, in a preferred embodiment the cellulolytic preparation comprising a polypeptide having cellulolytic enhancing activity (GH61A), preferably the one disclosed in WO 2005/074656. The cellulolytic preparation may further comprise a beta-glucosidase, such as a beta-glucosidase derived from a strain of the genus Trichoderma, Aspergillus or Penicillium, including the fusion protein having beta-glucosidase activity disclosed in WO 2008/057637 (Novozymes). in an embodiment the cellulolytic preparation may also comprises a CBH II, preferably Thielavia terrestris cellobiohydrolase II (CEL6A). In an embodiment the cellulolytic preparation also comprises a cellulase enzymes preparation, preferably the one derived from Trichoderma reesei or Humicola insolens.

The cellulolytic activity may. in a preferred embodiment, be derived from a fungal source, such as a strain of the genus Trichoderma, preferably a strain of Trichoderma reesei; or a strain of the genus Humicola, such as a strain of Humicola insolens; or a strain of Chrysosporium, preferably a strain of Chrysospohum lucknowense.

In an embodiment the cellulolytic enzyme preparation comprises a polypeptide having cellulolytic enhancing activity (GH61A) disclosed in WO 2005/074656: a cellobiohydrolase. such as Thielavia terrestris cellobiohydrolase II (CEL8A), a beta-glucosidase (e.g., the fusion protein disclosed in WO2008/057634) and cellulolytic enzymes, e.g., derived from Trichoderma reesei.

In an embodiment the cellulolytic enzyme preparation comprises a polypeptide having cellulolytic enhancing activity (GH61A) disclosed in WO 2005/074656; a beta-glucosidase (e.g., the fusion protein disclosed in WO 2008/057637) and cellulolytic enzymes, e.g., derived from Trichoderma reesei.

In an embodiment the cellulolytic enzyme composition is the commercially available product CELLUCLAST™ 1.5L. CELLUZYME™ (from Novozymes A/S, Denmark) or ACCELERASE™ 1000 (from Genencor Inc. USA).

A cellulase may be added for hydrolyzing the pre-treated lignocellulose-containing material. The cellulase may be dosed in the range from 0.1-100 FPU per gram total solids (TS), preferably 0.5-50 FPU per gram TS, especially 1-20 FPU per gram TS. In another embodiment at least 0.1 mg cellulolytic enzyme per gram total solids (TS), preferably at least 3 mg cellulolytic enzyme per gram TS, such as between 6 and 10 mg cellulolytic enzyme(s) per gram TS is(are) used for hydrolysis.

### Endoglucanase (EG)

Endoglucanases (EC No. 3.2.1.4) catalyses endo hydrolysis of 1,4-beta-D-glycosidic linkages in cellulose, cellulose derivatives (such as carboxy methyl cellulose and hydroxy ethyl cellulose), lichenin, beta-1,4 bonds in mixed beta-1,3 glucans such as cereal beta-D-glucans or xyloglucans and other plant material containing celluiosic parts. The authorized name is endo-1,4-beta-D-glucan 4-glucano hydrolase, but the abbreviated term endoglucanase is used in the present specification. Endoglucanase activity may be determined using carboxymethyl cellulose {CIVIC} hydrolysis according to the procedure of Ghose, 1987, Pure and Appl. Chem. 59: 257-268.

In a preferred embodiment endoglucanases may be derived from a strain of the genus Trichoderma, preferably a strain of Trichoderma reesei; a strain of the genus Humicola, such as a strain of Humicola insolens; or a strain of Chrysosporium, preferably a strain of Chrysosporium lucknowense.

### Cellobiohydrolase (CBH)

The term “cellobiohydrolase” means a 1,4-beta-D-glucan cellobiohydrolase (E.C. 3.2.1.91), which catalyzes the hydrolysis of 1,4-beta-D-glucosidic linkages in cellulose, cellooligosaccharides, or any beta-1,4-linked glucose containing polymer, releasing cellobiose from the reducing or non-reducing ends of the chain.

Examples of cellobiohydroloses are mentioned above including CBH I and CBH II from Trichoderma reseei; Humicola insolens and CBH II from Thielavia terrestris cellobiohydrolase (CELL6A)

Cellobiohydrolase activity may be determined according to the procedures described by Lever et al., 1972, Anal. Biochem. 47: 273-279 and by van Tilbeurgh et al., 1982, FEBS Letters 149: 152-156; van Tilbeurgh and Claeyssens, 1985, FEBS Letters 187: 283-288. The Lever et al. method is suitable for assessing hydrolysis of cellulose in corn stover and the method of van Tilbeurgh et al. is suitable for determining the cellobiohydrolase activity on a fluorescent disaccharide derivative.

### Beta-glucosidase

One or more beta-glucosidases (sometimes referred to as “cellobiases”) may be present during hydrolysis, SSF, or HHF.

The term “beta-glucosidase” means a beta-D-glucoside glucohydrolase (E.C. 3.2.1.21), which catalyzes the hydrolysis of terminal non-reducing beta-D-glucose residues with the release of beta-D-glucose. For purposes of the present invention, beta-glucosidase activity is determined according to the basic procedure described by Venturi et al., 2002, J. Basic Microbiol. 42: 55-66, except different conditions were employed as described herein. One unit of beta-glucosidase activity is defined as 1.0 μmole of p-nitrophenol produced per minute at 50° C., pH 5 from 4 mM p-nitrophenyl-beta-D-glucopyranoside as substrate in 100 mM sodium citrate, 0.01% TWEEN® 20.

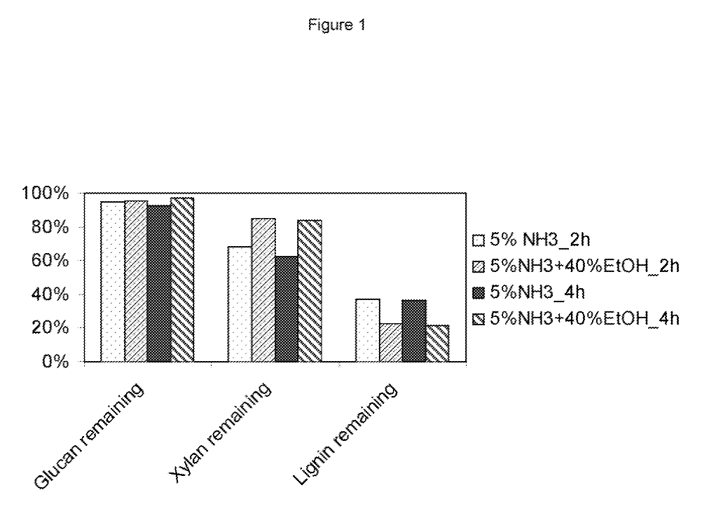

In a preferred embodiment the beta-glucosidase is of fungal origin, such as a strain of the genus Trichoderma, Aspergillus or Penicillium. In a preferred embodiment the beta-glucosidase is a derived from Trichoderma reesei, such as the beta-glucosidase encoded by the bgI1 gene (see FIG. 1 of EP 562003), in another preferred embodiment the beta-glucosidase is derived from Aspergillus oryzae (recombinantly produced in Aspergillus oryzae according to WO 02/095014), Aspergillus fumigatus (recombinantly produced in Aspergillus oryzae according to Example 22 of WO 02/095014) or Aspergillus niger (1981, J. Appl. 3: 157-163).

### Cellulolytic Enhancing Activity

The term “cellulolytic enhancing activity” is defined herein as a biological activity that enhances the hydrolysis of a lignocellulose derived material by proteins having cellulolytic activity. For purposes of the present invention, cellulolytic enhancing activity is determined by measuring the increase in reducing sugars or in the increase of the total of cellobiose and glucose from the hydrolysis of a lignocellulose derived material , e.g., pre-treated lignocellulose-containing material by cellulolytic protein under the following conditions: 1-50 mg of total protein/g of cellulose in PCS (pre-treated corn stover), wherein total protein is comprised of 80-99,5% w/w cellulolytic protein/g of cellulose in PCS and 0,5-20% w/w protein of cellulolytic enhancing activity for 1-7 day at 50° C. compared to a control hydrolysis with equal total protein loading without cellulolytic enhancing activity (1-50 mg of cellulolytic protein/g of cellulose in PCS).

The polypeptides having cellulolytic enhancing activity enhance the hydrolysis of a lignocellulose derived material catalyzed by proteins having cellulolytic activity by reducing the amount of cellulolytic enzyme required to reach the same degree of hydrolysis preferably at least 0.1-fold, more at least 0.2-fold, more preferably at least 0.3-fold, more preferably at least 0.4-fold, more preferably at least 0.5-fold, more preferably at least 1-fold, more preferably at least 3-fold, more preferably at least 4-fold, more preferably at least 5-fold, more preferably at least 10-fold, more preferably at least 20-fold, even more preferably at least 30-fold, most preferably at least 50-fold, and even most preferably at least 100-fold.

In a preferred embodiment the hydrolysis and/or fermentation is carried out in the presence of a cellulolytic enzyme in combination with a polypeptide having enhancing activity. In a preferred embodiment the polypeptide having enhancing activity is a family GH61A polypeptide. WO 2005/074647 discloses isolated polypeptides having cellulolytic enhancing activity and polynucleotides thereof from Thielavia terrestris. WO 2005/074656 discloses an isolated polypeptide having cellulolytic enhancing activity and a polynucleotide thereof from Thermoascus aurantiacus. U.S. Application Publication No. 2007/0077630 discloses an isolated polypeptide having cellulolytic enhancing activity and a polynucleotide thereof from Trichoderma reesei.

### Hemicellulolytic Enzymes

Hemicellulose can be broken down by hemicellulases and/or acid hydrolysis to release its five and six carbon sugar components.

In an embodiment of the invention the lignocellulose derived material may be treated with one or more hemicellulases.

Any hemicellulase suitable for use in hydrolyzing hemicellulose. preferably into xylose, may be used. Preferred hemicellulases include xylanases, arabinofuranosidases, acetyl xylan esterase, feruloyl esterase, glucuronidases, galactanase, endo-galactanase, mannases, endo or exo arabinases, exo-galactanses, pectinase, xyloglucanase, or mixtures of two or more thereof. Preferably, the hemicellulase for use in the present invention is an exo-acting hemicellulase, and more preferably, the hemicellulase is an exo-acting hemicellulase which has the ability to hydrolyze hemicellulose under acidic conditions of below pH 7, preferably pH 3-7, An example of hemicellulase suitable for use in the present invention includes VISCOZYME™ (available from Novozymes A/S, Denmark).

In an embodiment the hemicellulase is a xylanase. In an embodiment the xylanase may preferably be of microbial origin, such as of fungal origin (e.g., Trichoderma, Meripilus, Humicola, Aspergillus, Fusarium) or from a bacterium (e.g., Bacillus). In a preferred embodiment the xylanase is derived from a filamentous fungus, preferably derived from a strain of Aspergillus, such as Aspergillus aculeatus; or a strain of Humicola, preferably Humicola lanuginosa. The xylanase may preferably be an endo-1,4-beta-xylanase, more preferably an endo-1,4-beta-xylanase of GH10 or GH11. Examples of commercial xylanases include SHEARZYME™ and BIOFEED WHEAT™ from Novozymes A/S, Denmark.

Arabinofuranosidase (EC 3.2.1.55) catalyzes the hydrolysis of terminal non-reducing alpha-L-arabinofuranoside residues in alpha-L-arabinosides.

Galactanase (EC 3.2.1.89), arabinogalactan endo-1,4-beta-galactosidase, catalyses the endohydrolysis of 1,4-D-galactosidic linkages in arabinogalactans.

Pectinase (EC 3.2.1.15) catalyzes the hydrolysis of 1,4-alpha-D-galactosiduronic linkages in pectate and other galacturonans.

Xyloglucanase catalyzes the hydrolysis of xyloglucan.

The hemicellulase may be added in an amount effective to hydrolyze hemicellulose. such as, in amounts from about 0.001 to 0.5 wt. % of total solids (TS), more preferably from about 0.05 to 0.5 wt. % of TS.

Xylanases may be added in amounts of 0.001-1.0 g/kg DM (dry matter) substrate, preferably in the amounts of 0.005-0.5 g/kg DM substrate, and most preferably from 0.05-0.10 g/kg DM substrate.

### Other Enzymes

Other hydrolytic enzymes may also be present during hydrolysis, fermentation, SSF, or HHF. Contemplated enzymes include alpha-amylases; glucoamylases or another carbohydrate-source generating enzymes, such as beta-amylases, maltogenic amylases and/or alpha-glucosidases; proteases; or mixtures of two of more thereof.

### Xylose Isomerase

Xylose isomerases (D-xylose ketoisomerase) (E.C. 5.3.1.5.) are enzymes that catalyze the reversible isomerization reaction of D-xylose to D-xylulose. Some xylose isomerases also convert the reversible isomerization of D-glucose to D-fructose. Therefore, xylose isomarase is sometimes referred to as “glucose isomerase.”

A xylose isomerase used in a method or process of the invention may be any enzyme having xylose isomerase activity and may be derived from any sources, preferably bacterial or fungal origin, such as filamentous fungi or yeast. Examples of bacterial xylose isomerases include the ones belonging to the genera Streptomyces, Actinoptanes, Bacillus, Flavobacterium, and Thermotoga, including T. neapolitana (Vieille et al., 1995, Appl. Environ. Microbiol 61(5): 1867-1875) and T. maritime.

Examples of fungal xylose isomerases are derived species of Basidiomycetes.

A preferred xylose isomerase is derived from a strain of yeast genus Candida, preferably a strain of Candida boidinii, especially the Candida boidinii xylose isomerase disclosed by, e.g., Vongsuvanlert et al., 1988. Agric. Biol. Chem. 52(7); 1817-1824. The xylose isomerase may preferably be derived from a strain of Candida boidinii (Kioeckera 2201), deposited as DSM 70034 and ATCC 48180, disclosed in Ogata et al., Agric. Biol. Chem, 33: 1519-1520 or Vongsuvanlert et al., 1988, Agric. Biol. Chem. 52(2): 1519-1520.

In one embodiment the xylose isomerase is derived from a strain of Streptomyces, e.g., derived from a strain of Streptomyces murinus (U.S. Pat. No. 4.687,742): S. flavovirens, S. albus, S. achromogenus, S. echinatus, S. wedmorensis all disclosed in U.S. Pat. No. 3,616,221. Other xylose isomerases are disclosed in U.S. Pat. No. 3,622,463, U.S. Pat. No. 4,351,903, U.S. Pat. No. 4,137,126, U.S. Pat. No. 3,625,828. HU patent No. 12,415, DE patent 2,417,642, JP patent No. 69,28,473, and WO 2004/044129 each incorporated by reference herein.

The xylose isomerase may be either in immobilized or liquid form. Liquid form is preferred.

Examples of commercially available xylose isomerases include SWEETZYME™ T from Novozymes A/S, Denmark.

The xylose isomerase is added to provide an activity level in the range from 0.01-100 IGIU per gram total solids.

### Treating Solution

In this aspect the invention relates to treating solutions suitable for treating lignocellulose-containing material in accordance with the method and/or process of the invention.

According to the invention the treating solution for delignifying lignocellulose-containing material comprising;
i) from 1-30 wt. % delignificatson catalyst; and
ii) from 5-60 wt. % lignin solubilizing agent.

In an embodiment the treating solution is an aqueous solution. In a preferred embodiment the solution comprising from 30-50 wt. %, preferably around 40 wt. % lignin solubilizing agent and from 2-10 wt. %, preferably around 5 wt. % delignificafion catalyst.

Examples of delignificafion catalysts and lignin solubilizing agents can be found above.

### Use

In this aspect the invention relates to the use of treating solution of the invention for delignifying lignocellulose-containing material.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims, in the case of conflict, the present disclosure, including definitions will be controlling.

Various references are cited herein, the disclosures of which are incorporated by reference in their entireties.

### MATERIALS & METHODS

### Materials:

Aqueous ammonia was purchased from Fisher Scientific Inc, USA
Ethanol was purchased from Sigma, USA.

### Cellulolytic Preparation A:

Cellulolytic composition comprising a polypeptide having cellulolytic enhancing activity (GH61A) disclosed in WO 2005/074656; a beta-glucosidase (fusion protein disclosed in WO 2008/057637), and cellulolytic enzymes preparation derived from Trichoderma reesei. Cellulase preparation A is disclosed in co-pending application PCT/US2008/065417.

### Methods:

### Measurement of Cellulase Activity Using Filter Paper Assay (FPU Assay)

1. Source of Method
1.1 The method is disclosed in a document entitled “Measurement of Cellulase Activities” by Adney and Baker, 1996, Laboratory Analytical Procedure, LAP-006, National Renewable Energy Laboratory (NREL). It is based on the IUPAC method for measuring cellulase activity (Ghose, 1987, Measurement of Cellulase Activities, Pure & Appl. Chem. 59: 257-268.
2. Procedure
2.1 The method is carried out as described by Adney and Baker, 1996, supra, except for the use of a 96 well plates to read the absorbance values after color development, as described below.
2.2 Enzyme Assay Tubes:
2.2.1 A rolled filter paper strip (#1 Whatman; 1×6 cm; 50 mg) is added to the bottom of a test tube (13×100 mm).
2.2.2 To the tube is added 1.0 ml of 0.05 M Na-citrate buffer (pH 4.80).
2.2.3 The tubes containing filter paper and buffer are incubated 5 min. at 50′C. (±0.1° C.) in a circulating water bath.
2.2.4 Following incubation, 0.5 mL of enzyme dilution in citrate buffer is added to the tube. Enzyme dilutions are designed to produce values slightly above and below the target value of 2.0 mg glucose.
2.2.5 The tube contents are mixed by gently vortexing for 3 seconds,
2.2.6 After vortexing, the tubes are incubated for 60 minutes at 50° C. (±0.1° C.) in a circulating water bath.
2.2.7 Immediately following the 60 min. incubation, the tubes are removed from the water bath, and 3.0 ml of DNS reagent is added to each tube to stop the reaction. The tubes are vortexed 3 seconds to mix.
2.3 Blank and Controls
2.3.1 A reagent blank is prepared by adding 1.5 ml of citrate buffer to a test tube.
2.3.2 A substrate control is prepared by placing a rolled filter paper strip into the bottom of a test tube, and adding 1.5 ml of citrate buffer.
2.3.3 Enzyme controls are prepared for each enzyme dilution by mixing 1.0 mL of citrate buffer with 0.5 ml of the appropriate enzyme dilution.
2.3.4 The reagent blank, substrate control, and enzyme controls are assayed in the same manner as the enzyme assay tubes, and done along with them.
2.4 Glucose Standards
2.4.1 A 100 mL stock solution of glucose (10.0 mg/mL) is prepared, and 5 mL aliquots are frozen. Prior to use, aliquots are thawed and vortexed to mix.
2.4.2 Dilutions of the stock solution are made in citrate buffer as follows;
   G1=1.0 mL stock+0.5 mL buffer=6.7 mg/mL=3.3 mg/0.5 mL
   G2=0.75 mL stock+0.75 mL buffer=5.0 mg/mL=2.5 mg/0.5 mL
   G3=0.5 mL stock+1.0 mL buffer=3.3 mg/mL=1.7 mg/0.5 mL
   G4=0.2 mL stock+0.8 mL buffer=2.0 mg/mL=1.0 mg/0.5 mL
2.4.3 Glucose standard tubes are prepared by adding 0.5 mL of each dilution to 1.0 mL of citrate buffer.
2.4.4 The glucose standard tubes are assayed in the same manner as the enzyme assay tubes, and done along with them.
2.5 Color Development
2.5.1 Following the 60 min. incubation and addition of DNS, the tubes are all boiled together for 5 mins, in a water bath.
2.5.2 After boiling, they are immediately cooled in an ice/water bath.
2.5.3 When cool, the tubes are briefly vortexed, and the pulp is allowed to settle. Then each tube is diluted by adding 50 microL from the tube to 200 microL of ddH₂O in a 96-well plate. Each well is mixed, and the absorbance is read at 540 nm.
2.6 Calculations (examples are given in the NREL document)
2.6.1 A glucose standard curve is prepared by graphing glucose concentration (mg/0.5 mL) for the four standards (G1-G4) vs. A₅₄₀. This is fitted using a linear regression (Prism Software), and the equation for the Sine is used to determine the glucose produced for each of the enzyme assay tubes.
2.6.2 A plot of glucose produced (mg/0.5 ml) vs. total enzyme dilution is prepared, with the Y-axis (enzyme dilution) being on a log scale.
2.6.3 A line is drawn between the enzyme dilution that produced just above 2.0 mg glucose and the dilution that produced just below that. From this line, it is determined the enzyme dilution that would have produced exactly 2.0 mg of glucose.
2.6.4 The Filter Paper Units/mL (FPU/mL) are calculated as follows:
   FPU/ml=0.37/enzyme dilution producing 2.0 mg glucose

### EXAMPLES

### Example 1

### Delignification of Corn Stover

Corn stover obtained from Midwest, USA, was ground using a hammer mill equipped with a screen having 5-mm holes. The particles that passed the screen were used as the feedstock for the experiment.

The moisture content of the particles was 8.9 wt. %. Compositional analysts of the corn stover feedstock following the NREL Standard Biomass Analytical Procedures (www.nrel.gov/biomass/analytical_procedures.html) is shown in Table 1. The reactors used for the delignification treatment were autoclave reactors which were constructed out of 1.905 cm (0.75″) (Monel tubes sealed with 316 stainless steel caps), A sand bath was used to provide heating for the treatment. At the beginning of the experiment, 2.2 grams of corn stover particles (containing 2 grams of dry matter) and 18 grams of aqueous treating solution were loaded into each reactor. The corn stover was soaked in the reactors at room temperature for one hour before heat-up. Timing started once the reactors were submerged info the sand bath.

FIG. 1 summarizes the results from ammonia/ethanol treatment of corn stover at 140° C. Using 5 wt, % aqueous ammonia (NH₃)+40 wt. % ethanol, high degree of lignin removal was reached at small expense of carbohydrate loss. After two hours of cooking, only approximately 22% of initial lignin is left in the solids, while approximately 84% of initial xylan was recovered. In comparison, the treatment without ethanol addition resulted in a substrate containing approximately 37% of initial lignin and approximately 68% of initial xylan. Under all conditions in this experiment, the recovery of cellulose was constantly higher (approximately 95-97%).

TABLE 1
Compositional analysis of corn stover feedstock (w/w, dry basis)

| | |
|---|---|
| Glucan | 35.2% |
| Xylan | 21.2% |
| Galactan | 1.9% |
| Arabinan | 3.4% |
| Acid insoluble lignin | 18.7% |
| Acid soluble lignin | 1.0% |

### Example 2

### Delignification of Wheat Straw

The experiment in Example 1 was repeated, except that wheat straw was used instead of corn stover and the temperature was 120° C.

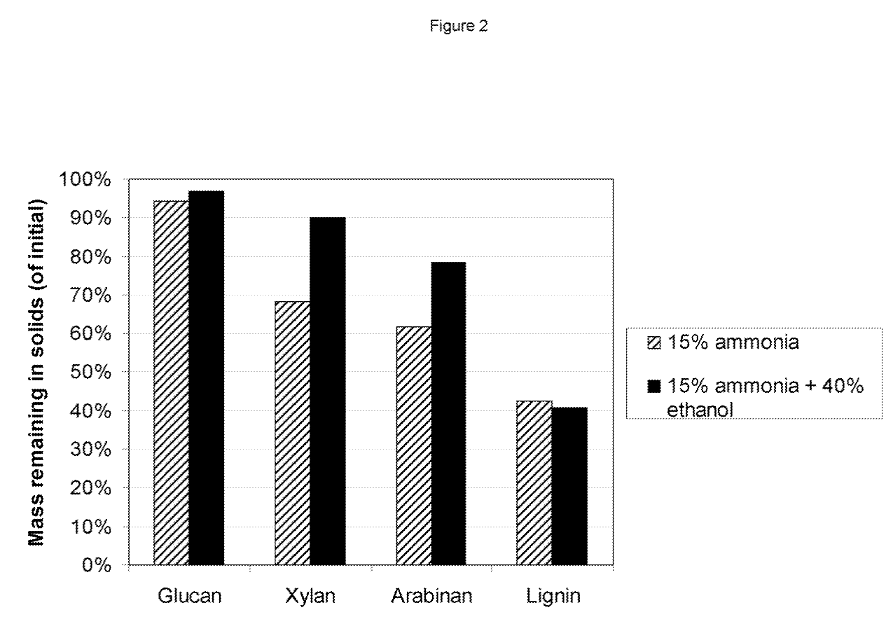

FIG. 2 shows the effect of ethanol addition on the carbohydrate and lignin remaining using wheat straw as the feedstock. The data indicated that ethanol addition improves the carbohydrate yields from the lignocellulose solids and promotes the removal of lignin.

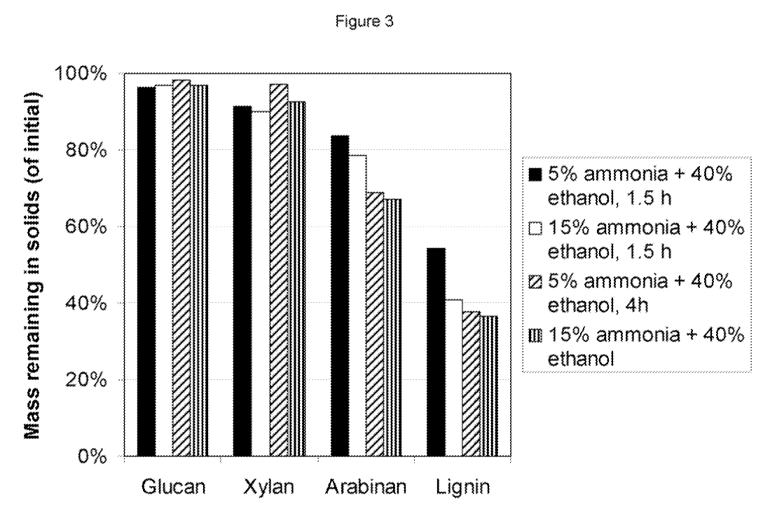

The effect of the ammonia concentration and processing time on carbohydrates and lignin remaining in the wheat straw is summarized in FIG. 3. For a processing time of 1.5 hours, increasing ammonia concentration from 5 wt. % to 15 wt. % significantly increased the removal of lignin; whereas, after 4 hours, the difference was insignificant. The delignification at 5 wt. % ammonia and 4 hours is slightly higher than that using 15 wt. % ammonia for 4 hours. Under all the conditions studied, the glucan and xylan retentions were above 95% and 90%, respectively.

### Example 3

### Delignification of Corn Stover and Enzymatic Hydrolysis

Corn stover obtained from Midwest was ground with a hammer mill and the particles passing through a screen with 2-mm pores were collected. The collected corn stover particles were washed with 50 volumes of tap wafer on a Whatman GF/D microfibre membrane and dried in a 50× oven until the moisture was below 5% (w/w). Compositional analysis of the corn stover feedstock was done following the NREL Standard Biomass Analytical Procedures (htfp://www.nrel.gov/biomass/analytical_procedures.html). The data are shown in Table 2.

TABLE 2
Composition of corn stover feedstock (w/w, dry basis)

| | |
|---|---|
| Glucan | 39.6% |
| Xylan | 21.5% |
| Galactan | 1.1% |
| Arabinan | 2.6% |
| Lignin | 20.1% |

Labmat reactors (1.75″ diameter×8″ length, Mathis, Model BFA-629/4) were used for the pretreatment. These reactors are cylindrical autoclaves made out of stainless steel. A sand bath was used to heat up the reactors. Prior to pretreatment, 10.45 grams of corn stover particles (containing 10 grams of dry matter) and 100 grams of NH₃/ethanol solution were loaded into each reactor. The corn stover was soaked in the sealed reactors at room temperature for 1.5 hours before heat-up. The sand bath was first heated up to 10° C. above the pre-treatment temperature and was reset to the pre-treatment temperature immediately before the reactors were submerged. Owing to the cool reactor bodies, the temperature of the sand bath dropped rapidly but stabilized at the set point within ten minutes. Timing started once the reactors were submerged in the sand bath. After the predetermined pre-treatment time, the reactors were taken out of the sand bath and quenched in cool water to stop the reaction. The cooled reactor was then opened and discharged. The pre-treated solids were then washed with de-ionized water on a Whatman GF/D membrane until pH was below 7.5.

Table 3 summarizes the composition and mass recoveries of the insoluble solids after pretreatment and water washing. The pre-treatment conditions were: solids/liquid (w/w)=10, 5% ammonia+40% ethanol (w/w), 130° C. and 3 hours. As can be seen, after the pre-treatment, only 24.5% of initial lignin was left in the solids, while 97.6% and 87.6% of initial glucan and xylan were recovered.

TABLE 3
Composition and mass recoveries of solids after pretreatment and
water washing

| | Composition | Mass recovery |
|---|---|---|
| | (w/w, dry basis) | (w/w) |
| Insoluble solids | — | 70.4% |
| Glucan | 55.0% | 97.6% |
| Xylan | 26.7% | 87.6% |
| Galactan | 1.0% | 60.7% |
| Arabinan | 2.8% | 76.6% |
| Lignin | 7.0% | 24.5% |

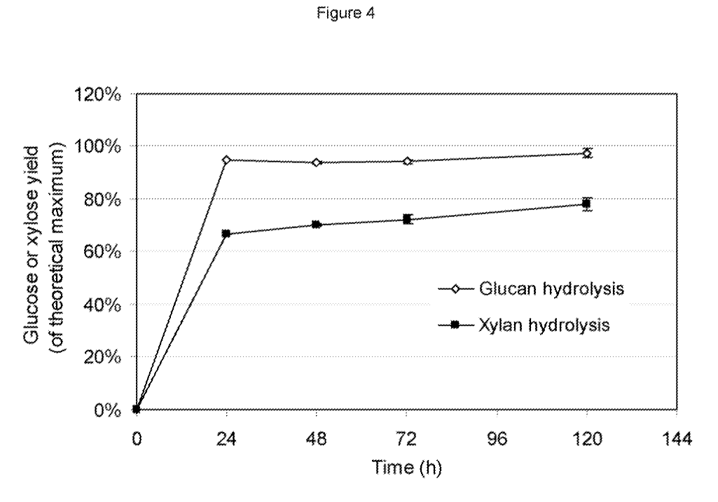

Pre-treated and washed solids were subjected to enzymatic hydrolysis by Cellulase Preparation A. The pre-treatment conditions were: solids-liquid ratio (w/w)=0.1, 5% (w/w) ammonia+40% (w/w) ethanol, 130° C., 3 h. Hydrolysis conditions: pH 4.8, 50° C., 150 rpm. Duplicates were run for the hydrolysis experiment. Hydrolysis was conducted in 125 ml shaking flasks. More than 95% of the glucan and 65% of xylan in the pre-treated corn stover were converted to glucose and xylose, respectively, within 24 hours. After 120 h, nearly 100% of the glucan and 80% of the xylan were hydrolyzed. The results are summarized in FIG. 4.

1-20. (canceled)

21. A method of delignifying lignocellulose-containing material, comprising treating the lignocellulose-containing material with a treating solution comprising a delignification catalyst and a lignin solubilizing agent.

22. The method of claim 21, wherein the delignification catalyst constitutes from 1-30 wt. % per g treating solution.

23. The method of claim 21, wherein the delignification catalyst is ammonium.

24. The method of claim 21, wherein the lignin solubilizing agent constitutes from 5-60 wt. % per g treating solution.

25. The method of claim 21, wherein the lignin solubilizing agent is ethanol, glycerol or acetone.

26. The method of claim 21, wherein treatment is carried out in a slurry wherein the lignocellulose-containing material constitutes from 5-30 wt. %.

27. The method of claim 21, wherein the temperature during treatment is in the range from 60-180° C.

28. The method of claim 21, wherein the treatment is carried out for 10 minutes to 1 week.

29. The method of claim 21, wherein delignification is carried out at alkaline pH.

30. The method of claim 21, wherein the lignocellulose-containing material is a non-wood material.

31. A process of producing a fermentation product from lignocellulose-containing material, comprising the steps of:
(a) delignifying lignocellulose-containing material using the method defined in claim 21;
(b) hydrolyzing the material;
(c) fermenting using a fermenting organism.

32. The process of claim 31, wherein steps (b) and (c) are carried out sequentially, simultaneously, or as a hybrid hydrolysis and fermentation process.

33. The process of claim 31, wherein the lignocellulose-containing material is treated with ammonium as delignification catalyst and ethanol as lignin solubilizing agent in step (a).

34. The process of claim 31, wherein remaining unsoluble lignin is removed after hydrolysis.

35. The process of claim 31, wherein hydrolysis in step (b) or SSF or HHF, is carried out using cellulolytic enzymes or hemicellulolytic enzymes, or a combination thereof.

36. The process of claim 31, wherein the fermenting organism in step (c) or SSF or HHF, is yeast.

37. The process of claim 31, wherein the fermentation product is ethanol or butanol.

38. A treating solution for delignifying lignocellulose-containing material comprising:
(a) from 1-30 wt. % delignification catalyst; and
(b) from 5-60 wt. % lignin solubilizing agent.

39. The treating solution of claim 38, wherein the delignification catalyst is ammonium, and the lignin solubilizing agent is ethanol, glycerol or acetone.
